(19) United States
(12) Patent Application Publication
(10) Pub. No.: US 2017/0305825 A1
(43) Pub. Date: Oct. 26, 2017

(54) IMPROVED METHOD FOR PREPARING PARA-THYMOL

(71) Applicant: LANXESS Deutschland GmbH, Cologne (DE)

(72) Inventors: Uwe BOEGER, Leverkusen (DE); Lutz HEUER, Dormagen (DE); Eberhard ZIRNGIEBL, Koeln (DE); Alexander HENSELER, Koeln (DE); Eszter FARKAS, Hilden (DE)

(73) Assignee: LANXESS Deutschland GmbH, Cologne (DE)

(21) Appl. No.: 15/516,763

(22) PCT Filed: Oct. 12, 2015
(86) PCT No.: PCT/EP2015/073538

§ 371 Date: Apr. 4, 2017

(30) Foreign Application Priority Data
Oct. 13, 2014 (EP) 14188640.8

(51) Int. Cl. C07C 37/84; C07C 39/06; C07C 37/14

(52) U.S. Cl. CPC C07C 37/84; C07C 37/14; C07C 39/06

(57) ABSTRACT

The present invention relates to an improved method for producing 4-isopropyl-3-methylphenol (p-thymol) from distillation residues of thymol production.

The present invention relates to an improved method for producing 4-isopropyl-3-methylphenol (p-thymol) from distillation residues of thymol production.

4-isopropyl-3-methylphenol is used, for example, as an antibacterial and microbicidal agent in cosmetics and mouthwashes with anti-plaque effect and also foot and hair care compositions having good skin compatibility. The preparation of 4-isopropyl-3-methylphenol is known in principle.

For instance, U.S. Pat. No. 3,331,879 describes the reaction of meta-cresol (m-cresol, 3-methylphenol) with propene over a zirconium catalyst, in which mainly thymol (2-isopropyl-5-methylphenol), but also many aromatic by-products, are formed. As a by-product, 4-isopropyl-3-methylphenol was identified in the reaction mixture with a content of 2% or 4.4% after a first distillation. The isolation of 4-isopropyl-3-methylphenol is not described.

DE 2139622 OS describes the occurrence of up to 19.5% of 4-isopropyl-3-methylphenol in the reaction of m-cresol with propene over an acidic zinc catalyst. The isolation of 4-isopropyl-3-methylphenol is also not described here.

DE 2528303 OS describes the occurrence of ca, 2% of 4-isopropyl-3-methylphenol in the reaction of meta-cresol with propene over a basic aluminum oxide catalyst. Isolation of 4-isopropyl-3-methylphenol from the diverse by-products is not described.

It is further known from U.S. Pat. No. 2,603,662 that 4-isopropyl-3-methylphenol can be obtained by a complex process as a by-product in the reaction of meta-cresol with isobutene.

It is common to all aforementioned methods that 4-isopropyl-3-methylphenol occurs as a secondary component in the alkylation of m-cresol with so many other secondary components that either recovery was not undertaken or is exceptionally cumbersome to carry out.

A process is known from DE 102007035515 A in which thymol and unreacted meta-cresol are firstly substantially removed by distillation from a reaction mixture of thymol production and the remaining residue is distilled in order to remove low-volatile or non-volatile substances, and the distillate thus obtained is crystallized after addition of up to 5% by weight water or the remaining residue is crystallized after addition of up to 5% by weight water and the crystallized residue is separated by distillation from low-volatile or non-volatile substances by distillation.

EP 2524906 A1 discloses a process in which thymol and unreacted meta-cresol are firstly substantially removed by distillation from a reaction mixture of thymol production. In a subsequent crystallization process, p-thymol is crystallized out and isolated from the distillation residue after slow cooling to room temperature with a content of 82 to 87% by weight. The p-thymol crude product is recrystallized in a further step with methylcyclohexane and activated carbon. After filtration and drying, white crystals of p-thymol are obtained with a purity of 99.8% by weight.

Therefore, a need exists to provide an improved method with which 4-isopropyl-3-methylphenol can be obtained cost-effectively in an efficient manner and in high purity and without recrystallization or washing with organic solvents.

A method has now been found for producing 4-isopropyl-3-methylphenol from reaction mixtures obtainable by reacting meta-cresol with propene in the presence of a catalyst, characterized in that at least
a) thymol and unreacted meta-cresol are largely removed from the reaction mixture by distillation, wherein a distillation residue is obtained, and
b) from the distillation residue remaining according to step a), a suspension comprising crystalline 4-isopropyl-3-methylphenol and a liquid phase is obtained by crystallization, and
c) from the suspension obtained according to step b), crystalline 4-isopropyl-3-methylphenol is separated from the liquid phase by the effect of centrifugal forces.

The method according to the invention preferably does not comprise a wash step, particularly preferably in step c), According to the invention, wash step is understood to mean treatment of the crystalline 4-isopropyl-3-methylphenol with solvents in which the crystalline 4-isopropyl-3-methylphenol itself is not dissolved, but in which only adhering impurities dissolve and are therefore removed from crystalline 4-isopropyl-3-methylphenol.

With the method according to the invention, an otherwise necessary costly and inconvenient purification of the 4-isopropyl-3-methylphenol by recrystallization from solvents to obtain high purity grades can be avoided.

The crystalline 4-isopropyl-3-methylphenol obtained according to step c) typically has a 4-isopropyl-3-methylphenol content of more than 90% by weight, preferably 91 to 99% by weight, particularly preferably 92 to 99% by weight, and especially preferably 92 to 96% by weight.

The residue up to 100% by weight typically comprises other products, such as in particular, further isomers of isopropylmethylphenol.

In one embodiment, the method according to the invention comprises the additional step of
d) recrystallization of the crystalline 4-isopropyl-3-methylphenol obtained from step c).

The recrystallization is preferably carried out using methylcyclohexane, particularly preferably in the presence of activated carbon.

With this step, the purity of the crystalline 4-isopropyl-3-methylphenol depending on the content obtained after step c) can be increased to more than 96, preferably from 97 to 100% by weight.

The scope of the invention comprises, in addition to the ranges and preferred ranges stated of formulae and parameters, also any combinations thereof, even if they are not fully explicitly listed below for practical reasons.

In the alkylation of meta-cresol with propene in the presence of a catalyst, which may be carried out in a manner known to those skilled in the art (see e.g. DE 3824284 OS or DE 2528303 OS), a reaction mixture is typically formed comprising, in addition to a major quantity of thymol, also about 1 to 3% by weight 4-isopropyl-3-methylphenol.

In a step a) of the method according to the invention, thymol and unreacted meta-cresol are substantially removed from the reaction mixture by distillation. The term substantially in this case means that the remaining residue has a proportion in total of thymol and meta-cresol taken together of 80% by weight or less, preferably 55% by weight or less and particularly preferably 30% by weight or less.

In this case, the distillation can be carried out in a manner known per se, for example, batchwise or continuously, wherein a continuous distillation under reduced pressure is preferred compared to standard pressure, which is for example 1 to 950 hPa, preferably from 50 to 950 hPa and particularly preferably from 50 to 150 hPa.

The temperature in the distillation at the top of the column is, for example, from 100 to 225° C. and preferably from 140 to 155° C. and the bottom temperature is, for example, from 120 to 260° C. and preferably from 170 to 190° C., in which it is clear to those skilled in the art that the temperatures in the distillation at the top of the column and at the bottom correlate with each other and also with the distillation pressure. Suitable distillation conditions can be readily ascertained by those skilled in the art.

The distillation is carried out preferably with the aid of a short-path evaporator, a column with or without internals, or by a falling film evaporator or also a thin-film evaporator. One theoretical plate is sufficient for the distillation. The use of more than one theoretical plate is naturally possible but not required.

The remaining residue typically comprises, in addition to 4-isopropyl-3-methylphenol, 20 to 30 other secondary components of low molecular weight structure, and also polymeric secondary components. After the distillation, the content of 4-isopropyl-3-methylphenol in the remaining usually black colored residue is typically 10 to 30% by weight.

The distillation residue remaining according to step a) preferably has a temperature from 120 to 180° C. and particularly preferably from 120 to 140° C.

According to step b), 4-isopropyl-3-methylphenol is crystallized from the distillation residue remaining according to step a), wherein a suspension comprising crystalline 4-isopropyl-3-methylphenol and a liquid phase is obtained.

Step b) can be carried out, preferably by cooling the distillation residue to temperatures, for example, from −20′ to 30° C., preferably to 0 to 30° C., and particularly preferably to 3 to 25° C. or ambient temperature.

In another embodiment, the crystallization is achieved by cooling the distillation residue remaining according to step a), wherein the temperature difference between the beginning and end of the crystallization process during cooling is for example 30K or more, preferably 40K or more, preferably from 40 to 100K.

In another embodiment of the method according to the invention, the crystallization may be induced or accelerated by seeding with crystalline 4-isopropyl-3-methylphenol.

The period of the crystallization is typically 5 to 100 hours, preferably 5 to 20 hours and particularly preferably 5 to 12 hours.

In step b), i.e. during the crystallization, the mixture is mechanically mixed or not, where mechanical mixing is preferred. This has the advantage in this case that smaller crystals are formed which can be removed more effectively from the liquid phase in the subsequent step c).

In the case of mechanical mixing, any device known for this purpose to those skilled in the art may be used. Examples include stirring devices, axial stirrers, radial stirrers and tangential stirrers. Stirring devices have at least one stirring element, for example a propeller, spiral or blade, which generate a flow of the liquid phase. Stirring devices also typically have a drive, e.g. a motor, and a connection between stirring element and drive, e.g. a shaft or a magnetic coupling. Depending on the stirrer type, flows are generated in a radial direction, i.e. orthogonal to the stirring axis, or in an axial direction, i.e. parallel to the stirring axis, or mixtures thereof. For example, paddle stirrers generate preferably radial flows, inclined blade stirrers and propeller stirrers generate axial flows. Axial flows can be directed upward or downward.

The suspension obtained according to step b) comprising crystalline 4-isopropyl-3-methylphenol and a liquid phase have a temperature of at most 30° C., preferably at most 25° C., particularly preferably from −10° to 25° C.

The suspension obtained according to step b) has a viscosity between 5° C. and 30° C. in a range from 20 to 250 MPas and has a density between 5 and 30° C. of 1 to 0.9 g/cm³, preferably from 0.98 to 0.95 g/cm³. The viscosity was determined by a capillary viscometer, the density by a helium comparison pycnometer.

In the suspension obtained according to step b), the 4-isopropyl-3-methylphenol is present predominantly crystalline with dimensions in the order of up to 800 μm, preferably from 300 to 500 μm, at a concentration of about 50 to 200 g/l, preferably from 70 to 180 g/l. In a preferred embodiment, 80% by weight or more of the 4-isopropyl-3-methylphenol present in the suspension are present as crystals with dimensions in the order of up to 800 μm, preferably from 300 to 500 μm, at a concentration of about 50 to 150 g/l. The crystals predominantly have a cubic form. Cubic crystal form according to the invention is defined as a crystal system which may be referred to as a three-dimensional axis intersection in which the angle of the axes are 90° with respect to each other, and in which all edges of the crystal are of equal length with a tolerance of 10%.

A portion of the liquid phase of the suspension obtained according to step b) may be separated, or not, by simple separation operations such as sedimentation and decanting, filtration or other simple solid-liquid separation operations known to those skilled in the art, which function by gravity. However, these simple separation operations are not sufficient to obtain the 4-isopropyl-3-methylphenol in the desired quality according to the invention.

In the subsequent step c), crystalline 4-isopropyl-3-methylphenol is separated from the liquid phase from the suspension obtained according to step b) by the effect of centrifugal forces.

Centrifugal forces according to the invention is defined as an inertial force which is directed radially outward from the rotation axis of a body. It is caused by the inertia of the body. The centrifugal force is derived from the centrifugal acceleration by multiplication with the mass.

In one embodiment in step c), the separation takes place by the action of pneumatic forces by means of a device which is suitable for a pressure of 2000 hPa or more which allows the liquid phase to pass and the purified 4-isopropyl-3-methylphenol to be retained. Such devices may be filter presses or continuous pressure filters.

In one embodiment in step c), the 4-isopropyl-3-methylphenol is separated from the liquid phase in centrifuges by the action of centrifugal forces. Centrifuges are categorized in the groups of sedimentation or filtration centrifuges. Both types are further subdivided into continuous and discontinuous centrifuges. Centrifuges typically have rotating elements, drums for example, such as in drum centrifuges, or buckets fixed on rotary axes, such as in filter bucket centrifuges, into which a solid-liquid mixture is introduced. By means of the centrifugal force resulting from rotation, the solid-liquid mixture is pressed against the outer wall of the rotating element. In the case of filtration centrifuges with drums as rotating element, a filter layer is located at the outer drum casing which retains the solid, whereas the liquid filtrate is spun outward through the pores or holes present in the filter layer. The filter layer used is preferably a textile filter layer. The drum can be banked vertically, such as in vertical centrifuges, or horizontally such as in horizontal centrifuges or scraper centrifuges. Horizontal or vertical centrifuges are discontinuous centrifuges since the filter cake deposited on the drum wall has to be removed in batches. Continuous filter centrifuges include, for example, pusher centrifuges, slide centrifuges or screen scroll centrifuges in which the deposited solid is continuously conveyed to an outlet and then out from the system, whereas the mixture to be separated is continuously conveyed into the system via an inlet. In sedimentation centrifuges, the solid present in the liquid is deposited by the action of a force field (generally centrifugal forces). The sediment that forms (slurry) and the clarified liquid (filtrate) can be drawn off either continuously or batchwise.

Scraper centrifuges represent a particular type of horizontal centrifuges. After completion of a centrifuge cycle, the solid is scraped using a scraping tool, a scraper knife for example, from the filter cake in a rotating drum and conveyed from the system, for example, by means of a discharge chute or a discharge screw.

The temperature of the suspension obtained according to step b) is maintained during step c) preferably at a maximum of 30° C., particularly preferably at a maximum of 25° C., especially preferably from −10° to 25° C.

Step c) of the method according to the invention is carried out, for example, in a centrifuge, preferably in a filter centrifuge, more preferably in a discontinuous filter centrifuge, or preferably in a horizontal centrifuge, particularly preferably in a scraper centrifuge. In one embodiment, in which step c) of the method according to the invention is carried out in one of the aforementioned centrifuges, the centrifuges have a drum diameter of 0.5 to 2 m, preferably from 0.8 to 1.5 m.

In one embodiment in step c) of the method according to the invention, centrifugal forces are used such that the suspension obtained according to step b) is subjected to a relative centrifugal acceleration (RCA) from 100 to 1000 g, preferably from 200 to 900 g, particularly preferably from 600 to 800 g. Appropriate devices as described above are sufficiently known to those skilled in the art.

Relative centrifugal acceleration is defined here as many times the average acceleration due to gravity. This can be calculated from the radius of the centrifuge drum and the rotation speed, for example, by the formula:

$$RCA = \frac{4 {π}^{2}}{g} {rn}^{2}$$

where g is the average acceleration due to gravity (9.80665 m/s²), r is the radius of the drum in meters and a is the rotational speed of the drum in s⁻¹.

The aforementioned centrifugal forces are applied preferably for a period of 5 to 30 minutes, preferably 10 to 25 minutes and particularly preferably 12 to 20 minutes.

In this case, preferably 80% by weight or more of the liquid phase, preferably 90% by weight or more of the liquid phase are separated from the crystalline 4-isopropyl-3-methylphenol.

The particular advantage, of the invention can be seen in that 41-isopropyl-3-methylphenol can be obtained efficiently and in exceptionally high purity as a secondary component in thymol production despite the presence of very many further secondary components.

### EXAMPLES

### Example 1

21.1 kg of a 60° C. warm, black distillation residue from the production of thymol by reacting m-cresol with propene in the presence of a catalyst, the distillation residue comprising 21.5% by weight thymol, 7.0% by weight 3-isopropyl-5-methylphenol, 25.0% by weight 4-isopropyl-3-methylphenol, 21.5% by weight 2,6-diisopropyl-3-methylphenol, 21.0% by weight 2,4-diisopropyl-5-methylphenol and a sum total of ca. 4% by weight, about 22 different otherwise alkylated cresols, were cooled slowly to room temperature with stirring in a 25-l capacity reactor over 16 hours, whereupon a suspension comprising crystalline 4-isopropyl-3-methylphenol and a liquid phase was formed. The precipitated 4-isopropyl-3-methylphenol was filtered off. 5.26 kg of 4-isopropyl-3-methylphenol were obtained with a purity of 83.4% by weight, which corresponded to a yield of 82.3% by weight of the 4-isopropyl-3-methylphenol present in the distillation residue.

### Example 2

A scraper centrifuge was employed using the following parameters:
Diameter of the drum: 1 m
Filter surface area: 1.57 m²
Fill volumes: 172 l
Edge height: 0.125 m
Drum length: 0.5 m

A suspension was used having the following parameters:
Temperature: 10° C.
Density: 0.97 g/cm³
Viscosity: 112.5 mPas

Concentration of
4-isopropyl-3-methylphenol: 150 g/l

The method according to the invention was carried out using the following parameters:
relative centrifugal acceleration: 700 g
suspension feed rate volumes: 120 l/min
suspension feed time: 180 s

After completion of the suspension feed, the relative centrifugal acceleration of the drum was increased for 900 s to 1000 g. Subsequently, the relative centrifugal acceleration of the drum was lowered to 45 g and the product was discharged using a scraper knife. 63 kg of crystalline 4-isopropyl-3-methylphenol were obtained which had a 4-isopropyl-3-methylphenol content of 96.1% by weight.

### Example 3 (Recrystallization)

580 g of methylcyclohexane and 5 g of activated carbon were added to 200 g of 4-isopropyl-3-methylphenol from Example 2 and heated under reflux for 10 minutes. The activated carbon is filtered off while hot and the filtrate is cooled to 25° C. with stirring at a cooling rate of 0.5 K/minute.

The 4-isopropyl-3-methylphenol that crystallizes out is filtered off and the crystals washed once with 100 g of methylcyclohexane on the nutsche filter. The solid filtered off is dried in the vacuum drying cabinet at 40° C./50 mbar.
Yield: 169 g of 4-isopropyl-3-methylphenol
Content: 99.9% by weight
Melting point: 11.5-112.5° C.

1. A method for producing 4-isopropyl-3-methylphenol from a reaction mixture obtained by reacting meta-cresol with propene in the presence of a catalyst, the method comprising:
a) removing thymol and unreacted meta-cresol from the reaction mixture by distillation, to produce a distillation,
b) from crystallizing 4-isopropyl-3-methylphenol from the distillation residue remaining according to step a) to form a suspension comprising crystalline 4-isopropyl-3-methylphenol and a liquid phase, and
c) separating the crystalline 4-isopropyl-3-methylphenol.

2. The method according to claim 1, further comprising:
d) recrystallizing the crystalline 4-isopropyl-3-methylphenol obtained from step c).

3. The method according to claim 1, wherein the separating in step c) is carried out by the effect of centrifugal forces in a centrifuge, preferably in a filter centrifuge, more preferably in a discontinuous filter centrifuge, or preferably in a horizontal centrifuge, particularly preferably in a scraper centrifuge.

4. The method according to claim 1, wherein the method does not include a step.

5. The method according to claim 1, wherein the crystalline 4-isopropyl-3-methylphenol obtained according to step c) has a 4-isopropyl-3-methylphenol content of more than 90% by weight, preferably 91 to 99% by weight, particularly preferably 92 to 99% by weight, and especially preferably 92 to 96% by weight.

6. The method according to claim 1, characterized in that step c) is carried out at a relative centrifugal acceleration from 100 to 1000 g, preferably from 200 to 900 g, particularly preferably from 600 to 800 g.

7. The method according to claim 1, wherein the separation c) comprises separation of the crystalline 4-isopropyl-3-methylphenol from the liquid phase by the effect of centrifugal forces.

8. The method according to claim 1, wherein the crystalline 4-isopropyl-3-methylphenol obtained according to step c) has a 4-isopropyl-3-methylphenol content of 92 to 99% by weight, and especially preferably 92 to 96% by weight

9. The method according to claim 1, wherein:
the separation c) comprises separation of the crystalline 4-isopropyl-3-methylphenol from the liquid phase by the effect of centrifugal forces at a relative centrifugal acceleration from 600 to 800 g; and
the crystalline 4-isopropyl-3-methylphenol obtained according to step c) has a 4-isopropyl-3-methylphenol content of 92 to 96% by weight.

10. The method according to claim 9, further comprising:
d) recrystallization of the crystalline 4-isopropyl-3-methylphenol obtained from step c using methylcyclohexane in the presence of activated carbon to produce 4-isopropyl-3-methylphenol having a purity of 97 to 100% by weight.
